(19) United States
(12) Patent Application Publication
(10) Pub. No.: US 2017/0239037 A1
(43) Pub. Date: Aug. 24, 2017

(54) TISSUE GRAFT FIXATION WITH TENSION ADJUSTMENT

(71) Applicant: Smith & Nephew, Inc., Memphis, TN (US)

(72) Inventors: Konsei Shino, Osaka (JP); Stephen Santangelo, Sturbridge, MA (US); Matthew Demmer, Memphis, TN (US)

(73) Assignee: Smith & Nephew, Inc., Memphis, TN (US)

(21) Appl. No.: 15/310,163

(22) PCT Filed: May 15, 2015
(86) PCT No.: PCT/US2015/031055

§ 371 Date: Nov. 10, 2016

Related U.S. Application Data
(60) Provisional application No. 61/993,336, filed on May 15, 2014.

(51) Int. Cl. A61F 2/08; A61B 17/17; A61B 17/064

(52) U.S. Cl. CPC A61F 2/0805; A61B 17/0642; A61B 17/1796; A61B 2017/0414

(57) ABSTRACT

Apparatus for tissue graft fixation. A tissue graft fixation device includes a main body part (15) provided with a pair of suture through holes (20, 25) positioned on opposite sides of a spherically radiused screw through hole (30), the pair of suture through holes sized to receive sutures, the spherically radiused screw through hole sized to receive a radiused head of a securing screw, and two spike parts (35, 40) extending substantially parallel to each other from an underside of the main body, the spike parts spaced apart at a predetermined distance slightly shorter than a diameter of a formed bone tunnel and adapted be stricken into a bone.

### CROSS REFERENCE TO RELATED APPLICATIONS

This application claims priority to U.S.Provisional Application No. 61/993,336, filed May 15, 2014, the entire contents of which is hereby incorporated by reference.

### BACKGROUND OF THE INVENTION

The present invention relates generally to anterior cruciate ligament (ACL) reconstruction, and specifically to tissue graft fixation with tension adjustment.

In general, anterior cruciate ligament (ACL) reconstruction involves replacing a torn ACL with tissue taken from a patient (autograft) or taken from a tissue donor (allograft). The procedure is usually done arthroscopically and involves making tunnels in the femur and tibia allowing passage of the graft material in order to reconstruct the ACL. During the procedure, there is typically an inherent graft weakness during a period after implantation when allograft and autograft tissues undergo necrosis, revascularization, and cellular proliferation. A graft fixation site for bone-tendon-bone (BTB) ACL reconstructions tends to be a weak link in the fixation construct and it is important to achieve strong fixation. Prior approaches have used interference screws, ENDOBUTTON® type devices, double spiked plates, and so forth, to achieve strong fixation.

What is needed is an apparatus that provides increased fixation strength while providing a way to adjust a tension of a graft at a time of fixation.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

The present invention is directed towards a method and an apparatus for tissue graft fixation with tension adjustment.

In an aspect, the invention features a tissue graft fixation device including a main body part provided with a pair of suture through holes positioned on opposite sides of a spherically radiused screw through hole, the pair of suture through holes sized to receive sutures, the spherically radiused screw through hole sized to receive a radiused head of a securing screw, and two spike parts extending substantially parallel to each other from an underside of the main body, the spike parts spaced apart at a predetermined distance slightly shorter than a diameter of a formed bone tunnel and adapted be stricken into a bone.

In another aspect, the invention features a method including providing a tendon graft comprised of tibial and femoral bone blocks each attached to a respective end of a tendon, each of the bone blocks made of a piece of bone, forming a femur-side bone tunnel in a femur and a tibia-side bone tunnel in a tibia, the tibia-side bone tunnel possessing an opening which opens to outside the tibia, threading sutures through the tibial and femoral bone blocks wherein ends of the threaded sutures are used for navigation and tensioning, providing a fixation device, the fixation device including a main body part provided with a pair of suture through holes positioned on opposites sides of a spherically radiused screw through hole, the pair of suture through holes sized to receive sutures, the spherically radiused screw through hole sized to receive a radiused head of a securing screw, and two spike parts extending substantially parallel to each other from an underside of the main body, the spike parts spaced apart at a predetermined distance slightly shorter than a diameter of a formed bone tunnel and adapted be stricken into a bone, positioning the fixation device approximately over a center of the tibia-side bone tunnel with a guide wire and impactor, the centering enabling the two spikes to engage the walls of the tunnel, using the impactor to fixate the fixation device into an approximate center of the bone, placing the screw over the guide wire, tensioning the graft using the suture ends and a tension gage, and articulating the screw to relieve the tensioned graft.

In another aspect, the invention features an apparatus including a drill guide configured to drill a guide wire into an approximate center of a bone block, the drill guide including a base supporting a drill channel, a bone graft secured to a distal end of the drill channel, wherein a first cannulated portion is placed within a second cannulated portion which centers the first cannulated portion through a center of the drill channel, the first cannulation guiding placement of the guide wire, the second cannulated portion guiding a cannulated drill over the previous placed guide wire to endure concentric location of each drill with respect to the bone block.

Embodiments of the invention may have one or more of the following advantages.

A tissue graft fixation device enables increased fixation strength while providing a manner to adjust a tension of a graft at the time of fixation during anterior cruciate ligament (ACL) reconstruction.

A screw through hole of the tissue graft fixation device is spherically radiused to receive a radiused head of a securing screw, which enables angulation of the securing screw.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

The invention will be more fully understood by reference to the detailed description, in conjunction with the following figures, wherein:

FIG. 1 is a front prospective of an exemplary tissue graft fixation device;

FIG. 2 is a cross section of the tissue graft fixation device of FIG. 1;

FIG. 3 illustrates a tibial tunnel created in a tibia;

FIG. 4 is an exemplary drill guide;

FIG. 5 illustrates a suture threaded through a tibial side of a bone block;

FIG. 6 illustrates a suture threaded through a femoral side of the bone block;

FIG. 7 illustrates positioning of a bone block;

FIG. 8 illustrates a centering of the tissue graft fixation device;

FIG. 9 illustrates fixation of the tissue graft fixation device into the bone; and

FIG. 10 illustrates a screw placed through a screw through hole of the tissue graft fixation device over a guide wire.

### DETAILED DESCRIPTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

In the description below, the term “or” is intended to mean an inclusive “or” rather than an exclusive “or.” That is, unless specified otherwise, or clear from context, “X employs A or B” is intended to mean any of the natural inclusive permutations. That is, if X employs A, X employs B, or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form.

Some embodiments may be described using the expression “one embodiment” or “an embodiment” along with their derivatives. These terms mean that a particular feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. These terms are not necessarily intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

The present disclosure will be used with reference to an anterior cruciate ligament (ACL) reconstruction, but it will be understood that the technology and methods of the present invention may have other applications for reconstruction of other parts of body. In addition, the present disclosure is used with reference to placement in a tibia, but it will be understood that the technology and methods of the present invention may used for femoral placement.

As fully described below, the tissue graft fixation device of the present invention is used in a ligament reconstruction operation to fix a tendon graft having a bone plug that can be inserted into a bone tunnel formed in a bone. In other words, the tissue graft fixation device of the present invention is used to fix a bone plug attached to at least one end of a tendon graft inside a bone tunnel formed at a portion where the tendon graft is to be transplanted and other end of the tendon graft is fixed. The tissue graft fixation device of the present invention is a tensile force-adjustable fixing tool.

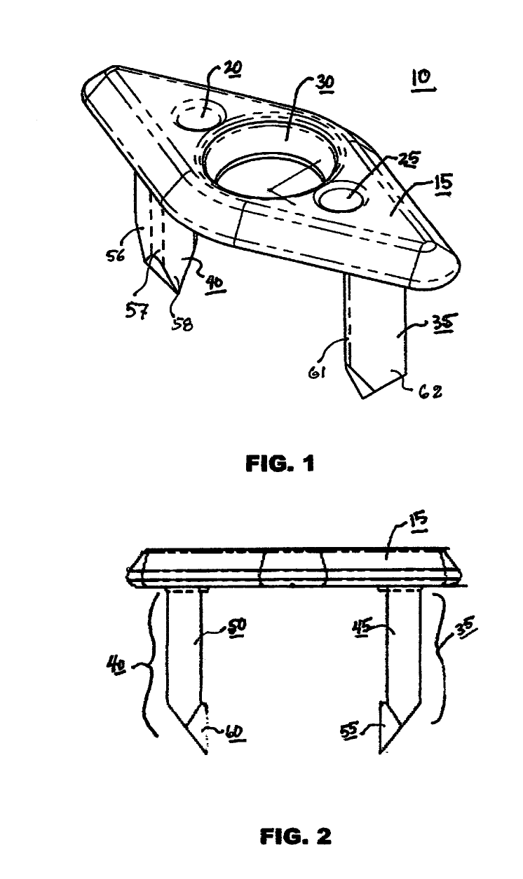

As shown in FIG. 1, a front prospective of an exemplary tissue graft fixation device 10 includes a main body part 15 provided with a pair of suture through holes 20, 25 positioned on opposites sides of a spherically radiused screw through hole 30. The pair of suture through holes 20, 25 are sized to receive sutures. The spherically radiused screw through hole 30 is sized to receive a radiused head of a securing screw, which enables angulation of the securing screw. If the screw through hole 30 was merely tapered and the head of the securing screw tapered also, no articulation of the screw would be possible. Two spike parts 35, 40 extend substantially parallel to each other from an underside of the main body 15. The spike parts 35, 40 are spaced apart at a predetermined distance slightly shorter than a diameter of a formed bone tunnel and can be stricken into a bone. More specifically, the spikes parts 35, 40 are not positioned fully in bone by an inserter, rather, portions of the spike parts 35, 40 are exposed within the formed bone tunnel. Further, a geometry of the spike parts 35, 40, fully described below, in combination with a screw placed into the screw through spherically radiused screw hole 30, enable piloting of the tissue graft fixation device 10 into a center of the formed bone tunnel.

The tissue graft fixation device 10 may be constructed from stainless steel, titanium, tungsten, a polymeric material and so forth.

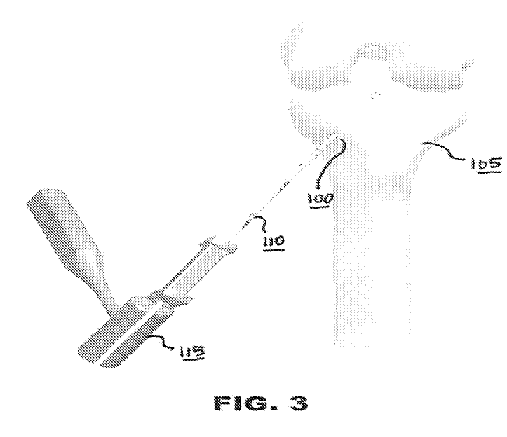

As shown in FIG. 2, the main body part 15 is formed in the shape of approximately a flat plate. In alternate embodiments, the main body part 15 may be formed in the shape of a bent plate or a curved plate. In a preferred embodiment, an overall length of the main body part is 18.2 mm (0.717 in.) and a thickness of the main body part is 1.5 mm (0.060 in.). In a preferred embodiment, a distance from the top of the main body part 15 to the bottom of each of the two spike parts 35, 40 is 8.5 mm (0.336 in.).

The two spike parts 35, 40 located on the underside of the main body part 15 are spaced at a predetermined interval shorter than a diameter of a formed bone tunnel. It is preferable that the interval between the spike parts 35, 40 is shorter than a diameter of an open portion of a bone tunnel to such an extent that the tissue graft fixation device 10 can be stricken into a bone, such as a tibia, while breaking a periphery of the open portion of the bone tunnel. It is more preferable that the interval between the spike parts 35, 40 is 0.5 mm (0.020 in.) shorter than the diameter of the bone tunnel. For example, if a diameter of a bone tunnel is 10 mm (0.394 in.), the interval between spike parts 35, 50 is preferred to be 9.5 mm (0.374 in.). In another example, if a diameter of a bone tunnel is 13 mm (0.512 in.), the interval between spike parts 35, 50 is preferred to be 12.5 mm (0.492 in.).

Each of the spike parts 35, 40 includes a main spike portion 45, 50, respectively, and a tapered tip portion 55, 60 respectively. In embodiments, the tapered tip portions 55, 60 are multifaceted. In one example, the tapered tip portions include faces 56, 57, 58, 61, 62, respectively, as shown in FIG. 1. In other embodiments, the numbers of faces can be more or less than the number illustrated in FIG. 1.

Referring again to FIG. 2, multifaceting of the tip portions 55, 60 enable better centering and engagement into the bone tunnel when the bone tunnel is the same size or slightly larger than the tip portions 55, 60. The main spike portions 45, 50 are positioned perpendicular to the main body part 15 and each of the tapered tip portions 55, 60 are tapered towards the bone tunnel. When the tissue graft fixation device 10 is inserted into the bone tunnel by an inserter, the spike parts 35, 40 do not reside 100% within the surrounding bone. Rather, a major portion of each of the spike parts 35, 40 resides within the surrounding bone while a minor portion of the spike parts 35, 40 penetrate into and reside in the bone tunnel. This configuration aids in centering the tissue graft fixation device 10 about the bone tunnel.

It is preferable that each of the tapered tip portions 55, 60 are positioned at approximately a forty-five (45) degree angle inwardly from each of the main spike portions 45, 50.

A method for tibial fixation of a BTB ACL reconstruction using the tissue graft fixation device will now be described.

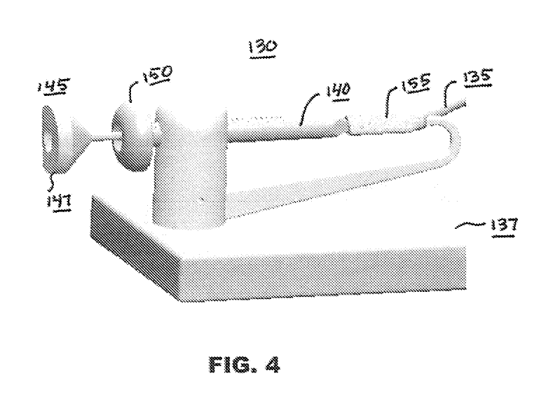

As shown in FIG. 3, a tibial tunnel 100 is created in a tibia 105 using a rigid rectangular dilator 110. A mallet 115 is used to advance the dilator 110 into the tibia 105 during tunnel 100 creation.

As shown in FIG. 4, a drill guide 130 is used to prepare a BTB graft 135. The drill guide 130 is used to drill a guide wire (not shown), e.g., 1 mm guide wire, into a center of the bone block.

The drill guide 130 includes a base 137 supporting a drill channel 140. The BTB graft 135 is secured to a distal end 145 of the drill channel. A first cannulated portion 147 is placed within a second cannulated portion 150, which centers the first cannulated portion 147 through a center of the drill channel 140 and thus into a center of the bone block 155, resulting in a centered hole in the bone block.

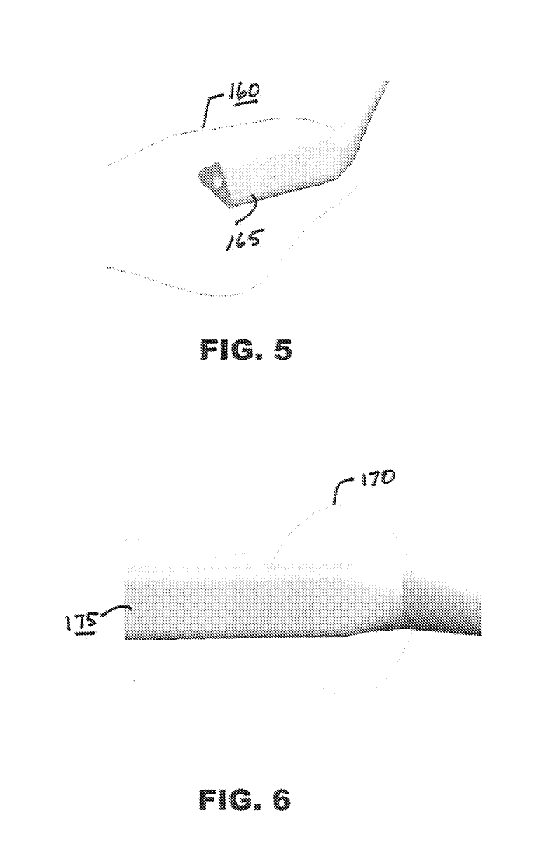

As shown in FIG. 5, a suture 160 is threaded through a tibial side 165 of the bone block. A shown in FIG. 6, a suture 170 is threaded through a femoral side 175 of the bone block in a quasi-figure eight orientation so that when the bone block is pulled into a femoral tunnel the suture 170 assists in piloting the bone block as it wraps around both sides of the bone block. More specifically, applying tension on an upper suture end causes the bone block to move in one direction while applying tension on a lower suture end causes the bone bock to move in an opposite direction. Thus, quasi-figure eight orientation of the suture 170 aids in navigation.

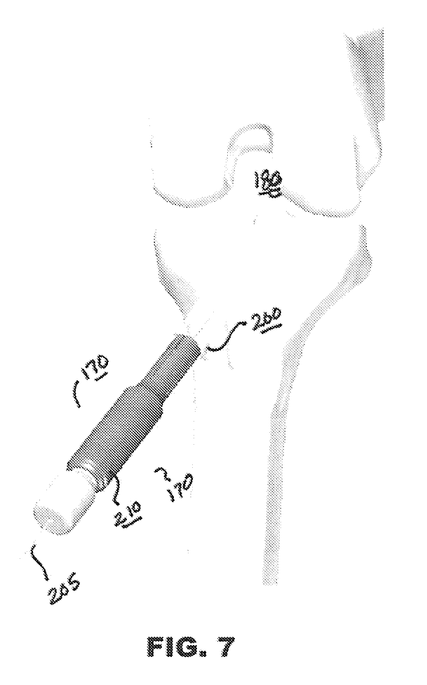

As shown in FIG. 7, the femoral side of the bone block 180 is placed within a femoral tunnel and the tibial side of the bone block 180 is placed within the tibial tunnel. The two ends of suture 170 (of FIG. 6) are threaded through suture through hole 20 (of FIG. 1) and suture through hole 25 (of FIG. 1), respectively, and a screw is placed within the spherically radiused screw through hole 30 (of FIG. 1). The tissue graft fixation device 200 is positioned within a center of the tibial tunnel using the guide wire 205 and an impactor tool 210 for guidance. An end of the impactor tool 210 is recessed to prevent damaging the two ends of suture 170 exposed through suture through hole 25 and suture through hole 25, along with a head of the screw, from being damaged. In addition, the impactor tool 210 is cannulated and centering. Thus, the impactor tool 210 is used to fixate the fixation device 200 into a center of the bone, i.e., the impactor tool 210 engages with a center of the spherically radiused screw through hole 30 (of FIG. 1).

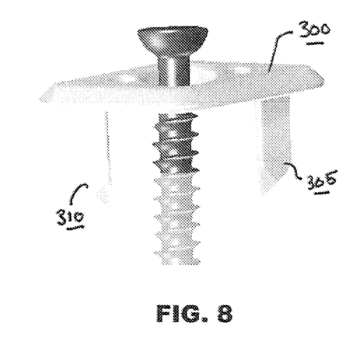

As shown in FIG. 8, centering the tissue graft fixation device 300 over the tibial bone tunnel enables tapered tip portions 305, 310, to engage the walls of the tibial bone tunnel, providing optimal fixation.

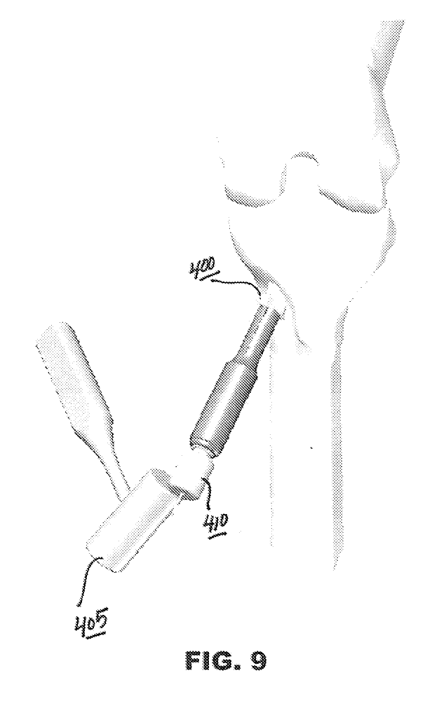

As shown in FIG. 9, once the tissue graft fixation device 400 is in position, a mallet 405 is used to strike an impactor 410 to fixate the tissue graft fixation device 200 into the bone. Once complete, the impactor 410 is removed.

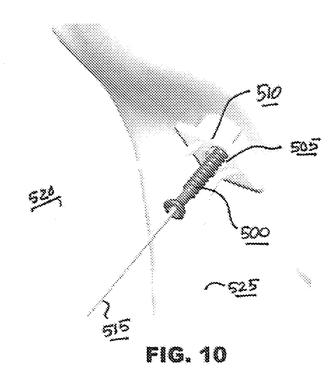

As shown in FIG. 10, a screw 500 is placed through a through hole 505 of the tissue graft fixation device 510 over a guide wire 515 The graft is tensioned with a sufficient force, such as 40 N, using the suture ends 520, 525 and a tension gauge (not shown). The screw 500 is then tightened until the tension on the suture ends 520, 525 is reduced to near 0 N, securing the tissue graft fixation device 510 in place. At this time, the screw is received in the spherically radiused recess 30 (of FIG. 1). The mating spherical geometry affords initial articulation of the screw with respect to the tissue graft fixation device thus maximizing compression, graft tension and resistance to the screw pulling through the plate. The suture ends 520, 525 may then be tied over the tissue graft fixation device 510 to provide increased fixation. As a result, the graft is anatomically fixated on the tibia 530.

While this invention has been particularly shown and described with references to preferred embodiments thereof, it will be understood by those skilled in the art that various changes in form and details may be made therein without departing from the spirit and scope of the present application as defined by the appended claims. Such variations are intended to be covered by the scope of this present application. As such, the foregoing description of embodiments of the present application is not intended to be limiting. Rather, any limitations to the invention are presented in the following claims.

What is claimed is:

1. A tissue graft fixation device comprising:
a main body part provided with a pair of suture through holes positioned on opposite sides of a spherically radiused screw through hole, the pair of suture through holes sized to receive sutures, the spherically radiused screw through hole sized to receive a radiused head of a securing screw; and
two spike parts extending substantially parallel to each other from an underside of the main body, the spike parts spaced apart at a predetermined distance slightly shorter than a diameter of a formed bone tunnel and adapted be stricken into a bone.

2. The tissue graft fixation device of claim 1 wherein the radiused head of the securing screw is spherically radiused.

3. The tissue graft fixation device of claim 1 wherein a geometry of the two spike parts and the screw placed into the spherically radiused screw through hole enable piloting into the formed bone tunnel.

4. The tissue graft fixation device of claim 1 wherein the main body part is formed in a shape of approximately a flat plate.

5. The tissue graft fixation device of claim 1 wherein the main body part and the two spike parts are constructed from a material selected from the group consisting of stainless steel, titanium, tungsten and a polymeric material.

6. The tissue graft fixation device of claim 1 wherein each of the two spike parts comprises:
a main spike portion; and
a tapered tip portion.

7. The tissue graft fixation device of claim 6 wherein the tapered tip portion is multifaceted.

8. The tissue graft fixation device of claim 6 wherein the tapered tip portion is positioned facing an interior of the formed bone tunnel at a 45 degree angle.

9. The tissue graft fixation device of claim 1 wherein the spherically radiused screw through hole enables angulation of the securing screw.

10. A method comprising:
providing a tendon graft comprised of tibial and femoral bone blocks each attached to a respective end of a tendon, each of the bone blocks made of a piece of bone;
forming a femur-side bone tunnel in a femur and a tibia-side bone tunnel in a tibia, the tibia-side bone tunnel possessing an opening which opens to outside the tibia;
threading sutures through the tibial and femoral bone blocks wherein ends of the threaded sutures are used for navigation and tensioning;
providing a fixation device, the fixation device comprising a main body part provided with a pair of suture through holes positioned on opposites sides of a spherically radiused screw through hole, the pair of suture through holes sized to receive sutures, the spherically radiused screw through hole sized to receive a radiused head of a securing screw, and two spike parts extending substantially parallel to each other from an underside of the main body, the spike parts spaced apart at a predetermined distance slightly shorter than a diameter of a formed bone tunnel and adapted be stricken into a bone;
positioning the fixation device approximately over a center of the tibia-side bone tunnel with a guide wire and impactor, the centering enabling the two spikes to engage the walls of the tunnel;
using the impactor to fixate the fixation device into an approximate center of the bone; placing the screw over the guide wire;
tensioning the graft using the suture ends and a tension gage; and
articulating the screw to relieve the tensioned graft.

11. The method of claim 10 wherein providing the tendon graft comprises:
engaging a drill guide to drill a guide wire into an approximate center of the bone block, the drill guide including a base supporting a drill channel;
securing the tendon graft to a distal end of the drill channel;
placing a first cannulated portion within a second cannulated portion; and
centering the first cannulated portion through a center of the drill channel and into the approximate center of the bone block,
the first cannulation guiding placement of a guide wire, the second cannulated portion guiding a cannulated drill over the previous placed guide wire to endure concentric location of each drill with respect to the bone block.

12. The method of claim 10 wherein the impactor engages with a center of the spherically radiused screw through hole.

13. The method of claim 12 wherein the impactor is cannulated and centering.

14. The method of claim 10 further comprising tying the ends of the sutures over the fixation device to provide increased fixation.

15. An apparatus comprising:
a drill guide configured to drill a guide wire into an approximate center of a bone block, the drill guide comprising a base supporting a drill channel, a bone graft secured to a distal end of the drill channel, wherein a first cannulated portion is placed within a second cannulated portion which centers the first cannulated portion through a center of the drill channel, the first cannulation guiding placement of the guide wire, the second cannulated portion guiding a cannulated drill over the previous placed guide wire to endure concentric location of each drill with respect to the bone block.

16. The apparatus of claim 15 wherein the guide wire is a one millimeter (1 mm) guide wire and the drill in two millimeters (2 mm).
